(19) United States
(12) Patent Application Publication
(10) Pub. No.: US 2022/0023160 A1
(43) Pub. Date: Jan. 27, 2022

(54) OIL-WATER SEPARATING COMPOSITION AND ARTICLE

(71) Applicant: Shiseido Company, Ltd., Tokyo (JP)

(72) Inventor: Tomoyuki KARIYA, Tokyo (JP)

(73) Assignee: Shiseido Company, Ltd., Tokyo (JP)

(21) Appl. No.: 17/296,929

(22) PCT Filed: Nov. 1, 2019
(86) PCT No.: PCT/JP2019/043087

§ 371 Date: May 25, 2021

(30) Foreign Application Priority Data
Nov. 26, 2018 (JP) 2018-220596

(51) Int. Cl. A61K 8/03; A61Q 1/14; A61Q 19/10; A61K 8/34

(52) U.S. Cl. CPC A61K 8/03; A61Q 1/14; A61K 2800/882; A61K 8/34; A61K 2800/87; A61Q 19/10

(57) ABSTRACT

An oil-water separating composition has an aqueous phase and an oil phase separated from the aqueous phase in a stationary state. The oil-water separating composition contains from 0.05 to 5% by mass of a polyol derivative with respect to the mass of the composition. The polyol derivative is at least one of a glycerin derivative and a glycol derivative.

### RELATED APPLICATION

The present application is based upon and claims the benefit of priority from Japanese Patent Application No. 2018-220596 filed on Nov. 26, 2018, the entire contents of which are incorporated herein by reference.

### TECHNICAL FIELD

The present disclosure relates to an oil-water separation-type composition in which the oil phase and the aqueous phase are separated in a stationary state. The present disclosure also relates to an article containing the aforementioned composition.

### BACKGROUND ART

Oil-water separation-type cosmetics (cleansing agents), wherein the oil phase and the aqueous phase are separated into two layers in a stationary state, are known in the art (e.g., Patent Literature 1). Such oil-water separation-type cosmetics are used in a temporarily emulsified state, which is created by a user shaking the container. Leaving the container to stand after use causes the emulsified state to be relieved, and thereby the oil phase and the aqueous phase revert to their separated state.

### CITATION LIST

### Patent Literature

Patent Literature 1: Japanese Unexamined Patent Publication No. 2001-213724A

### SUMMARY OF INVENTION

### Technical Problem

The following analysis can be made from the perspective of the present disclosure.

As described above, in cases of using an oil-water separating composition as disclosed in Patent Literature 1, it is necessary to form a temporarily emulsified state. Therefore, to enable a user to confirm that an emulsified state has been formed at the time of use, a transparent container is typically used for housing the oil-water separating composition. Further, a lightweight resin (e.g., polyester resin) is typically used for the container to enable even a female user to easily shake the container containing liquid.

When shaking is performed to create a temporarily emulsified state, the content comes into contact with the inner surface of the resin container. With conventional oil-water separating compositions, the content that has come into contact with the container's inner surface remains as liquid droplets (liquid beads) on the container's entire inner surface above the liquid surface. These liquid droplets do not disappear simply by being left to stand, but will remain on the container's inner wall surface over a long period of time. Since the container is transparent, the liquid droplets will be seen through the container, thus impairing the appearance of the product including the container.

Further, when the container is left to stand after temporary emulsification, the oil phase and the aqueous phase of the oil-water separating composition will re-separate. If, however, re-separation is insufficient, the interface between the oil phase and the aqueous phase becomes unclear, which will also impair the appearance of the product.

In cases where the oil-water separating composition is employed, for example, as a cosmetic product, the product's aesthetic appearance is also deemed important. To allow a product to keep an excellent aesthetic appearance, there is a demand for an oil-water separating composition that does not leave liquid droplets adhering to the container's inner surface for a long time and that provides a clear interface between the oil phase and the aqueous phase after emulsification.

### Solution to Problem

According to a first aspect of the present disclosure, an oil-water separating composition is provided, the composition comprising an aqueous phase, and an oil phase separated from the aqueous phase in a stationary state. The composition contains from 0.06 to 1.8% by mass of a polyol derivative with respect to the mass of the composition. The polyol derivative is a glycerin derivative represented by Chem. 1 and/or a glycol derivative represented by Chem. 2.

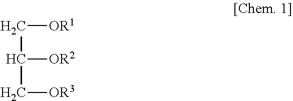

In the chemical formula represented by Chem. 1, one of R¹, R², and R³ is a C₄₋₁₅ alkyl group, alkenyl group or acyl group, and two are each a hydrogen atom.

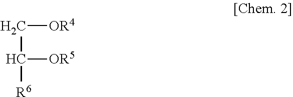

In the chemical formula represented by Chem. 2, one of R⁴ and R⁵ is a C₁₀₋₂₀ alkyl group, alkenyl group or acyl group, and the other is a hydrogen atom. R⁶ is a C₁₋₄ alkyl group, alkenyl group or acyl group, or a hydrogen atom.

According to a second aspect of the present disclosure, an article is provided, the article comprising the composition according to the first aspect, and a container that houses the composition. At least a portion of the container has transparency enabling an interior of the container to be visible.

### Advantageous Effects of Invention

The oil-water separating composition of the present disclosure inhibits liquid droplets from remaining on the inner wall surface of a container. Also, the interface between the oil phase and the aqueous phase becomes clear at the time of re-separation of the oil phase and the aqueous phase after temporary emulsification. Thus, the container containing the oil-water separating composition can maintain its excellent appearance.

### BRIEF DESCRIPTION OF DRAWINGS

FIG. 1 is a photograph of a sample which is an example of a criterion for evaluating remaining liquid droplets in the Test Examples.

FIG. 2 is a photograph of a sample which is an example of a criterion for evaluating remaining liquid droplets in the Test Examples.

FIG. 3 is a photograph of a sample which is an example of a criterion for evaluating remaining liquid droplets in the Test Examples.

FIG. 4 is a photograph of a sample which is an example of a criterion for interface evaluation in the Test Examples.

FIG. 5 is a photograph of a sample which is an example of a criterion for interface evaluation in the Test Examples.

FIG. 6 is a photograph of samples according to Test Examples 1 to 3.

FIG. 7 is a photograph of samples according to Test Examples 1, 3, and 4 after being shaken.

FIG. 8 is a photograph of samples according to Test Examples 5, 7, and 8 after being shaken.

FIG. 9 is a photograph of samples according to Test Examples 6, 9, and 10 after being shaken.

FIG. 10 is a photograph of samples according to Test Examples 1 and 11 to 13 after being shaken.

FIG. 11 is a photograph of samples according to Test Examples 2, 3, 14, and 15 after being shaken.

### DESCRIPTION OF EMBODIMENTS

Preferred modes according to the aforementioned aspects will be described below.

According to a preferred mode of the above first aspect, the oil-water separating composition further comprises from 0.01 to 0.1% by mass of an alkylbetaine-type surfactant.

According to a preferred mode of the above first aspect, a content by percentage of a surfactant is 0.2% by mass or less with respect to the mass of the composition.

According to a preferred mode of the above first aspect, the oil-water separating composition further comprises from 0.1 to 5% by mass of a salt with respect to the mass of the composition.

According to a preferred mode of the above first aspect, the glycerin derivative includes ethylhexylglycerin and/or hexylglycerol.

According to a preferred mode of the above first aspect, the glycol derivative includes at least one selected from the group consisting of propylene glycol laurate, propylene glycol stearate, and propylene glycol isostearate.

According to a preferred mode of the above first aspect, a content by percentage of the oil phase is from 20 to 80% by mass with respect to the mass of the composition. A content by percentage of the aqueous phase is from 20 to 80% by mass with respect to the mass of the composition.

According to a preferred mode of the above first aspect, the oil-water separating composition is to be used by being formed into a temporarily emulsified state by a user.

According to a preferred mode of the above first aspect, the oil-water separating composition is a cosmetic.

According to a preferred mode of the above first aspect, the oil-water separating composition is a cosmetic cleansing agent.

According to a preferred mode of the above first aspect, the oil-water separating composition is a leave-on-type composition.

According to a preferred mode of the above second aspect, the container comprises a polyester.

In the following description, POE is an abbreviation of polyoxyethylene, and POP is an abbreviation of polyoxypropylene. The number in parentheses after POE or POP indicates the average number of moles of POE groups or POP groups added in the compound in question.

In the present disclosure, “substantial amount” refers to an amount capable of bringing about effects due to addition of the compound in question.

An oil-water separating composition according to a first embodiment of the present disclosure will be described below.

The oil-water separating composition according to the first embodiment includes a liquid-state oil phase and a liquid-state aqueous phase. In a stationary state where the oil-water separating composition has been left to stand for a sufficient length of time (i.e., when not in use), a primary portion of the oil phase and a primary portion of the aqueous phase are separated into two, upper and lower layers (i.e., are not emulsified).

The oil-water separating composition according to the first embodiment contains a polyol derivative. The polyol derivative contains a glycerin derivative and/or a glycol derivative.

For the glycerin derivative, it is possible to use, for example, an alkylglyceryl ether and/or a glycerin ester, and particularly a monoalkylglyceryl ether. The glycerin derivative may be, for example, a compound represented by Chem. 3. In the chemical formula represented by Chem. 3, any one of R¹, R², and R³ may be a C₄₋₁₅ alkyl group, alkenyl group or acyl group, and any two may each be a hydrogen atom. The alkyl group, alkenyl group or acyl group may be linear or may be branched. It is preferred that the alkyl group, alkenyl group or acyl group contains 4 or more carbon atoms. The number of carbon atoms in the alkyl group, alkenyl group or acyl group may preferably be 15 or fewer, more preferably 12 or fewer.

Examples of the glycerin derivative may include ethylhexylglycerin (octoxyglycerin), hexylglycerin, glyceryl isooctanoate, polyglyceryl-2 laurate, glyceryl monooctanoate, and the like. Among the above, from the viewpoint of eliminating liquid droplets adhering to the inner surface of a container, it is preferred to use ethylhexylglycerin having a 2-ethylhexyl group and/or hexylglycerin having a hexyl group. An example of a commercially available product of ethylhexylglycerin may include Sensiva SC50 (from Schulke & Mayr GmbH).

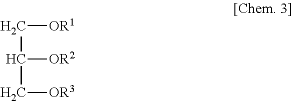

For the glycol derivative, it is possible to use, for example, a glycol ester and/or a glycol ether. For the glycol derivative, it is possible to use, for example, a propylene glycol fatty acid ester and/or a propylene glycol ether, and particularly a propylene glycol mono-fatty acid ester. For the glycol derivative, it is possible to use one of compounds represented by Chem. 4. In the chemical formula represented by Chem. 4, one of R⁴ and R⁵ may be a C₁₀₋₂₀ alkyl group, alkenyl group or acyl group, and the other may be a hydrogen atom. R⁶ may be a C₁₋₄ alkyl group, alkenyl group or acyl group, or a hydrogen atom. The alkyl group, alkenyl group or acyl group may be linear or may be branched.

Examples of the glycol derivative may include propylene glycol laurate, propylene glycol stearate, propylene glycol isostearate, and the like.

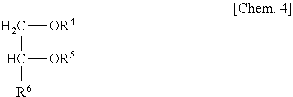

The glycerin derivative and the glycol derivative may be present in either the oil phase or the aqueous phase.

The content by percentage of the polyol derivative with respect to the mass of the composition is preferably 0.06% by mass or greater, more preferably 0.07% by mass or greater, further preferably 0.08% by mass or greater. The content by percentage of the polyol derivative with respect to the mass of the composition may be 0.1% by mass or greater, 0.2% by mass or greater, or 0.5% by mass or greater. If the content of the polyol derivative is less than 0.06% by mass, the aforementioned actions may not be obtained sufficiently. The content by percentage of the polyol derivative with respect to the mass of the composition is preferably 1.8% by mass or less, more preferably 1.5% by mass or less, further preferably 1.2% by mass or less. The content by percentage of the polyol derivative with respect to the mass of the composition may be 1% by mass or less, 0.8% by mass or less, or 0.5% by mass or less. If the content of the polyol derivative exceeds 1.8% by mass, the interface between the oil phase and the aqueous phase may become unclear.

By blending the polyol derivative, it is possible to inhibit the oil-water separating composition from continuing to adhere, as liquid droplets, to the inner surface of a container in a space above the liquid. Even if liquid droplets are formed on the inner wall of a container, the liquid droplets can be made to disappear spontaneously in a short time. Thus, it is possible to inhibit liquid droplets adhering to the inner surface of a container from being seen through the container, and thereby improve the product's aesthetic appearance. The polyol derivative's effect of eliminating liquid droplets is thought to be particularly effective in resin-made containers, and particularly containers containing polyester resins such as polyethylene terephthalate (PET).

The polyol derivative also acts to clearly define the interface between the oil phase and the aqueous phase at the time of re-separation of the oil phase and the aqueous phase that occurs by leaving the composition to stand in a stationary state after temporary emulsification. In this way, the appearance of the oil-water separating composition as seen through a container can be further improved.

In cases where the oil-water separating composition is a cleansing agent, a compound represented by Chem. 3 can also improve cleansability.

The oil phase of the oil-water separating composition according to the first embodiment can be set as appropriate depending on the purpose of the oil-water separating composition. For example, the oil phase may be an oily component capable of dissolving oil-soluble components blended to the oil-water separating composition. In cases where the oil-water separating composition is to be used as a cleansing agent, the oil phase can serve as an oily component useful for removing objects to be cleansed off (e.g., cosmetics). Preferably, the oil phase is liquid at room temperature.

Examples of oily components in the oil phase may include liquid oils, solid fats, waxes, hydrocarbons, higher fatty acids, higher alcohol, synthetic ester oils, and silicone oils.

Examples of the liquid oil that may be used may include avocado oil, camellia oil, turtle oil, macadamia nut oil, corn oil, mink oil, olive oil, rapeseed oil, egg yolk oil, sesame oil, par chic oil, wheat germ oil, southern piece oil, castor oil, linseed oil, safflower oil, cotton seed oil, perilla oil, soybean oil, groundnut oil, brown real oil, torreya oil, rice bran oil, Chinese tung oil, Japanese tung oil, jojoba oil, germ oil, triglycerol, and the like.

Examples of the solid fat that may be used may include cacao butter, coconut oil, horse fat, hydrogenated coconut oil, palm oil, beef tallow, sheep tallow, hydrogenated beef tallow, palm kernel oil, lard, beef bones fat, Japan wax kernel oil, hardened oil, hoof oil, Japan wax, hydrogenated caster oil, and the like.

Examples of the waxes that may be used may include beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether, and the like.

Examples of the hydrocarbon oils that may be used may include liquid paraffin, ozocerite, squalane, pristane, paraffin, ceresin. squalene, vaseline, microcrystalline wax, n-hexane, isohexane, cyclehexane, n-octane, isooctane, n-nonane, n-decane, isododecane, isohexadecane, and the like.

Examples of the higher fatty asid that may be used may include lauric acid, myristic acid, palmitic acid, stearic acid, behenic acid, oleic acid, undecylenic acid, tallic acid, isostearic acid, linoleic acid, linolenic acid, eicosapentaenoic acid (EPA), docosahexaenoic acid (DHA) and the like.

Examples of the higher alcohol that may be used may include linear alcohol (such as lauryl alcohol, cetyl alcohol, stearyl alcohol, behenyl alcohol, myristyl alcohol, oleyl alcohol, and cetostearyl alcohol); branched-chain alcohol (such as monostearylglycerin ether (batyl alcohol), 2-decyltetradecinol, lanolin alcohol, cholesterol, phytosterol, hexyldodecanol, isostearyl alcohol, and octyldodecanol) and the like.

Examples of the synthesis ester oils that may be used may include isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxy stearate, ethylene glycol di-2-ethyl hexanoate, di-penta erythritol fatty acid ester, N-alkyl glycol monoisostearate, neopentyl glycol dicaprate, diisostearyl malate, glyceryl di-2-heptyl undecanoate, trimethyrol propane tri-2-ethyl hexanoate, trimethyrol propane triisostearate, pentaerythritol tetra-2-ethyl hexanoate, glyceryl tri-2-ethyl hexanoate, glyceryl trioctanoate, glyceryl triisopalmitate, trimethyrol propane triisostearate, cetyl 2-ethylhexanoate, 2-ethylhexyl palmitate, glyceryl trimyristate, glyceride tri-2-heptyl undecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, and the like.

Examples of the silicone oil may include silicone compounds such as dimethylpolysiloxane, methylhydrogenpolysiloxane, methylphenylpolysiloxane, stearoxymethylpolysiloxane, polyether-modified organopolysiloxane, fluoroalkyl/polyoxyalkylene co-modified organopolysiloxane, alkyl-modified organopolysiloxane, terminal-modified organopolysiloxane, fluorine-modified organopolysiloxane, amino-modified organopolysiloxane, silicone gel, acrylic silicone, trimethylsiloxysilicic acid, silicone RTV rubber and the like.

Among the aforementioned oily components, hydrocarbon oils, ester oils, silicone oils, and the like are preferable from the viewpoint of cleansability against oily cosmetics.

The content by percentage of the oil phase with respect to the mass of the composition is preferably 20% by mass or greater, more preferably 30% by mass or greater, further preferably 40% by mass or greater. The quantitative balance with the amount of the aqueous phase may be impaired if the content of the oil phase is less than 20% by mass. The content by percentage of the oil phase with respect to the mass of the composition may be, for example, preferably 80% by mass or less, more preferably 70% by mass or less, further preferably 60% by mass or less. The quantitative balance with the amount of the aqueous phase may be impaired if the content of the oil phase exceeds 80% by mass.

The aqueous phase contains water. Water to be used in the aqueous phase may be water used in products such as cosmetics and quasi-pharmaceutical products, with examples including purified water, ion-exchanged water, tap water, etc.

The aqueous phase may further include a water-soluble alcohol. Examples of the water-soluble alcohol may include at least one selected from, for example, lower alcohols, polyols, polyol polymers, divalent alcohol alkyl ethers, divalent alcohol alkyl ethers, divalent alcohol ether esters, glycerin monoalkyl ethers, sugar alcohols, monosaccharides, oligosaccharides, polysaccharides, and derivatives thereof.

Examples of the lower alcohol may include ethanol, propanol, isopropanol, isobutyl alcohol, t-butyl alcohol, and the like.

Examples of the polyhydric alcohol may include dihydric alcohol (such as ethylene glycol, propylen glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc); trihydric alcohol (such as glycerin, trimethylolpropane, etc); tetrahydric alcohol (such as such as pentaerythritol such as 1,2,6-hexanetriol, etc); pentahydric alcohol (such as xylitol, etc); hexahydric alcohol (such as sorbitol, mannitol, etc); polyhydric alcohol polymer (such as diethylene glycol, dipropylene glycol, triethylene glycol, polypropylene glycol, tetraethylene glycol, diglycerin, polyethylene glycol, triglycerin, tetraglycerin, polyglycerin, etc); dihydric alcohol alkyl ethers (such as ethylene glycol monomethyl ether, ethylene glycol monoethyl ether, ethylene glycol monobutyl ether, ethylene glycol monomphenyl ether, ethylene glycol monohexyl ether, ethylene glycol mono2-methylhexyl ether, ethylene glycol isoamyl ether, ethylene glycol benzil ether, ethylene glycol isopropyl ether, ethylene glycol dimethyl ether, ethylene glycol diethyl ether, ethylene glycol dibutyl ether, etc); dihydric alcohol alkyl ethers (such as diethylene glycol monomethyl ether, diethylene glycol monoethyl ether, diethylene glycol monombutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc); dihydric alcohol ether ethers (such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disaccinate, diethylene glycol mono ethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc); glycerin monoalkyl ether (such as chimyl alcohol, selachyl alcohol, batyl alcohol, etc); sugar alcohol (such as sorbitol, maltitol, maltotriose, mannitol, sucrose, erythritol, glucose, fructose, starch sugar, maltose, xylitol, starch sugar hydrogenated alcohol, etc); glycolide, tetrahydrofurfuryl alcohol; POE-tetrahydrofurfuryl alcohol; POP/POE-butyl ether; tripolyoxypropylene glycerin ether; POP-glycerin ether; POP-glycerin ether phosphoric acid; POP/POE-pentaerythritol ether; polyglycerin, and the like.

Examples of the monosaccharides may include at least one selected from triose (such as D-glyceryl aldehyde, dihydroxyacetone, etc); tetrose (such as D-erythrose, D-erythrulose, D-threose, erythritol, etc); pentaose (such as L-arabinose, D-xylose, L-lyxose, D-arabinose, D-ribose, D-ribulose, D-xylulose, L-xylulose, etc); hexalose (such as D-glucose, D-talose, D-psicose, D-galactose, D-fructose, L-galactose, L-mannose, D-tagatose, etc); heptose (such as aldoheptose, heptulose, etc); octose (such as octulose, etc); deoxy sugar (such as 2-deoxy-D-ribose, 6-deoxy-L-galactose, 6-deoxy-L-mannose, etc); amino sugar (such as D-glucosamine, D-galactosamine, sialic acid, amino uronic acid, muramic acid, etc); uronic acid (such as D-grucuronic acid, D-mannuronic acid, L-guluronic acid, D-garacturonic acid, L-iduronic acid, etc) and the like.

Examples of the oligosaccharide may include at least one selected from sucrose, guntianose, umbelliferose, lactose, planteose, isolignoses, α,α-trehalose, raffinose, lignoses, umbilicin, stachyose, verbascoses, and the like.

Examples of the polysaccharide may include at least one selected from cellulose, quince seed, chondroitinsulfate, starch, galactan, dermatan sulfate, glycogen, acasia gum, heparansulfate, hyaluronan, gum tragacanth, keratan sulfate, chondoroitin, xanthan gum, mucoitin sulfate, guar gum, dextran, keratosulfate, locust bean gum, succinoglycan, caronic acid, and the like.

Examples of other polyols may include at least one polyol selected from polyoxyethylene methyl glucoside (Glucam E-10), polyoxypropylene methyl glucoside (Glucam P-10), and the like.

Among the aforementioned water-soluble alcohols, it is preferred to employ ethanol, butylene glycol, dipropylene glycol, or the like, from the viewpoint of emulsification adjustability and preservative properties.

The content by percentage of the aqueous phase with respect to the mass of the composition is preferably 20% by mass or greater, more preferably 30% by mass or greater, further preferably 40% by mass or greater. The quantitative balance with the amount of the oil phase may be impaired if the content of the aqueous phase is less than 20% by mass. The content by percentage of the aqueous phase with respect to the mass of the composition may be, for example, preferably 80% by mass or less, more preferably 70% by mass or less, further preferably 60% by mass or less. The quantitative balance with the amount of the oil phase may be impaired if the content of the aqueous phase exceeds 80% by mass.

The oil-water separating composition may further contain a surfactant. The surfactant may be added to bring the oil phase and the aqueous phase into a temporarily emulsified state by shaking at the time of use. In cases where the oil-water separating composition is a cleansing agent, the surfactant may be added to improve cleansability. Examples of surfactants may include the following surfactants.

Anionic Surfactant:

Examples of the anionic surfactants that may be used may include fatty acid soap (such as sodium laurate, and sodium palmitate); higher alkyl sulfate ester salt (such as sodium lauryl sulfate, and potassium lauryl sulfate); alkyl ether sulfate ester salt (such as POE-lauryl sulfate triethanolamine, and sodium POE-lauryl sulfate); N-acyl sarcosinic acid (such as sodium lauroyl sarcocinate); higher fatty acid amide sulfonate (such as sodium N-stearoyl-N-methyltaurate, sodium N-myristoyl-N-methyltaurate, sodium methyl cocoyl taurate, and sodium laurylmethyl taurate); phosphate ester salt (sodium POE-oleylether phosphate, POE-stearylether phosphate, potassium cetyl phosphate); sulfosuccinate (such as sodium di-2-ethylhexyl sulfosuccinate, sodium monolauroyl monoethanolamide polyethylene sulfosuccinate, and sodium lauryl polypropylene glycol sulfosuccinate); alkylbenzene sulfonate (such as sodium linear dodecylbenzene sulfonate, triethanolamine linear dodeylbenzene sulfonate, and linear dodecylbenzene sulfonate); higher fatty acid ester sulfate ester salt (such as sodium hydrogenated gryceryl cocoate sulfate); N-acyl glutamate (such as monosodium N-lauroyl glutamate, disodium N-stearoyl glutamate, and monosodium N-myristoyl-L-glutamate); sulfonated oil (such as Turkey red oil); POE-alkyl ether carboxylic acid; POE-alkyl aryl ether carboxylate; α-olefine sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; sodium casein; and the like.

Cationic Surfactant:

Examples of the cationic surfactants may include alkyltrimethyl ammonium salt (such as stearyltrimethyl ammonium chloride, lauryltrimethyl ammonium chloride); alkylpyridinium salt (such as cetylpyridinium chloride); dialkyldimethyl ammonium salt (such as distearyldimethyl ammonium chloride); poly (N,N′-dimethyl-3,5-methylenepiperidinium) chloride; alkyl quaternary ammonium salt; alkyldimethylbenzyl ammonium salt; alkylisoquinolinium salt; dialkylmorphonium salt; POE alkylamine; alkylamine salt; polyamine fatty acid derivative; amyl alcohol fatty acid derivative; benzalkonium chloride; benzethonium chloride, amino acid-based cationic surfactant (such as ethyl L-cocoyl arginine DL-pyrrolidonecarboxylic acid salt) and the like.

Amphoteric Surfactant:

Examples of the amphoteric surfactant that may be used may include: imidazoline-based amphoteric surfactant (such as sodium 2-undecyl-N,N,N-(hydroxyethylcarboxymethyl)-2-imidazoline and 2-cocoyl-2-imidazolinium hydroxide-1-carboxyethyloxy disodium salt); and betaine-based surfactant (such as 2-heptadecyl-N-carboxymethyl-N-hydroxyethyl imidazolinium betaine, lauryl dimethylaminoacetic acid betaine, alkyl betaine, amidobetaine, and sulfobetaine).

Hydrophilic Nonionic Surfactant:

Examples of the hydrophilic nonionic surfactants that may be used may include POE sorbitan fatty acid ester (such as POE sorbitan monooleate, POE sorbitan monostearate, POE sorbitan monooleate, POE sorbitan tetraoleate); POE sorbit fatty acid ester (such as POE sorbit monolaurate, POE sorbit monooleate, POE sorbit pentaoleate, POE sorbit monostearate), POE glyceryl fatty acid ester (such as POE monooleate such as POE glyceryl monostearate, POE glyceryl monoisostearate, POE glyceryl triisostearate); POE fatty acid ester (such as POE distearate, POE monodioleate, ethyleneglycol distearate); POE alkyl ether (such as POE lauryl ether, POE oleyl ether, POE stearyl ether, POE behenyl ether, POE-2-octyldodecyl ether, POE cholestanol ether); puluronic type (such as Puluronic), POE/POP alkyl ethers (such as POE/POP cetyl ether, POE/POP 2-decyltetradecyl ether, POE/POP monobutyl ether, POE/POP hydrogenated lanoline, POE/POP glycerin ether); tetra POE/tetra POP ethylenediamine condensation products (such as Tetronic); POE castor oil hydrogenated castor oil derivative (such as POE caster oil, POE hydrogenated caster oil, POE hydrogenated caster oil monoisostearate, POE hydrogenated castor oil triisostearate, POE hydrogenated caster oil monopyroglutamate monoisostearate diester, POE hydrogenated oil maleate); POE beeswax/lanoline derivative (such as POE sorbitol beeswax); alkanolamide (such as coconut oil fatty acid diethanolamide, lauric acid monoethanolamide, fatty acid isopropanolamide); POE propyleneglycol fatty acid ester; POE alkyl amines; POE fatty acid amide; sucrose fatty acid ester; alkylethoxydimethylamine oxide; trioleyl phosphoric acid and the like.

Lipopholic Nonionic Surfactant:

Examples of the lipophilic nonionic surfactants may include sorbitan fatty acid ester (such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2 ethylhexylate, diglycerol sorbitan tetra-2 ethylhexylate, etc); glyceryl polyglyceryl fatty acid (such as glyceryl monocotton oil fatty acid, glyceryl monoerucate, glyceryl sesquioleate, glyceryl monostearate, glyceryl a, a′-oleate pyroglutamate, glyceryl monostearate malate, etc); propylene glycol fatty acid ester (such as propylene glycol monostearate, etc); hydrogenated caster oil derivative; glyceryl alkyl ether, and the like.

In cases where the oil-water separating composition is to be used as a leave-on-type skin cleanser, it is even preferred that the surfactant is, for example, alkyl betaine, ethyl L-cocoyl arginine DL-pyrrolidonecarboxylic acid salt (PCA ethyl cocoyl arginate), benzalkonium chloride, or the like, from the viewpoint of causing less irritation to the skin.

The content by percentage of the surfactant with respect to the mass of the composition is preferably 0.02% by mass or greater, more preferably 0.05% by mass or greater, further preferably 0.07% by mass or greater. If the content of the surfactant is less than 0.02% by mass, a temporarily emulsified state may not be formed. The content by percentage of the surfactant with respect to the mass of the composition is preferably 0.3% by mass or less, more preferably 0.2% by mass or less, further preferably 0.15% by mass or less. If the content by percentage of the surfactant exceeds 0.3% by mass, the emulsified state may not be relieved after temporary emulsification by shaking, and the composition may not return to its oil-water two-layer state. Also, in cases where the oil-water separating composition is to be used as a leave-on-type cleansing agent for the skin, the composition may become highly irritant to the skin and may also cause stickiness.

The oil-water separating composition may further contain a salt.

The salt may be an inorganic salt or an organic salt. Examples of salts may include sodium chloride, potassium chloride, sodium citrate, sodium edetate, and the like.

The content by percentage of the salt with respect to the mass of the composition is preferably 0.1% by mass or greater, more preferably 0.2% by mass or greater, further preferably 0.3% by mass or greater. If the content of component (G) is less than 0.1% by mass, the aforementioned actions may not be obtained sufficiently. The content by percentage of the salt with respect to the mass of the composition is preferably 2% by mass or less, more preferably 1.5% by mass or less, further preferably 1% by mass or less. If the content of the salt exceeds 2% by mass, emulsification after shaking may be insufficient in cases where the composition is an oil-water two-layer separation-type composition.

The oil-water separating composition may further contain a trialkylamine oxide. The trialkylamine oxide may be added to remove, from the skin, dyes staining the skin. For example, the trialkylamine oxide may be added to remove acidic dyes that bond with proteins in the skin by chemical interaction (e.g., ionic interaction). The trialkylamine oxide can be used as a leave-on-type cleansing agent that does not need to be rinsed off.

The trialkylamine oxide may be water-soluble or water-insoluble (oil-soluble). The trialkylamine oxide may be a mixture of a water-soluble trialkylamine oxide and an oil-soluble trialkylamine oxide. Solubility to water or oily components can be adjusted by adjusting the length of the alkyl group(s) in the trialkylamine oxide.

The trialkylamine oxide may be dissolved in either the oil phase or the aqueous phase. For example, in cases where the trialkylamine oxide is oil-soluble, the trialkylamine oxide can be added to the aqueous phase by being dissolved in a water-soluble alcohol.

The trialkylamine oxide may have a structure represented by Chem. 5. R⁷, R⁸, and R⁹ may each be a linear alkyl group or a branched-chain alkyl group. For example, any two of R⁷, R⁸, and R⁹ (e.g., R⁷ and R⁸) may each be a C₁₋₄ alkyl group. The remaining one of R⁷, R⁸, and R⁹ (e.g., R⁹) may be a C₁₂₋₂₆ alkyl group. The C₁₂₋₂₆ alkyl group may be, for example, at least one selected from a dodecyl group (lauryl group), an octadecyl group (stearyl group), and a decyltetradecyl group. For example, in the trialkylamine oxide, R⁷ and R⁸ may each be a methyl group, as represented by Chem. 6, and R⁹ may be a decyltetradecyl group.

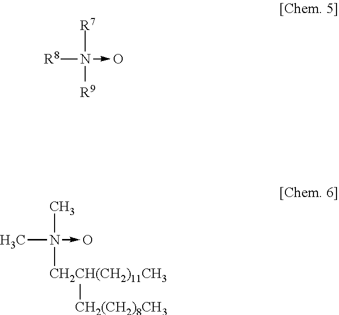

The content by percentage of the trialkylamine oxide with respect to the mass of the composition is preferably 0.01% by mass or greater, more preferably 0.02% by mass or greater, further preferably 0.05% by mass or greater, further preferably 0.08% by mass or greater, further preferably 0.1% by mass or greater, further preferably 0.12% by mass or greater, further preferably 0.15% by mass or greater, further preferably 0.18% by mass or greater. If the content of component (A) is less than 0.01% by mass, the cleansability against dye components may deteriorate. The content by percentage of the trialkylamine oxide with respect to the mass of the composition may be, for example, 2% by mass or less, 1% by mass or less, 0.8% by mass or less, 0.6% by mass or less, or 0.4% by mass or less.

If necessary, the oil-water separating composition of the present disclosure may contain other components as appropriate, such as powder bodies, moisturizers, water-soluble polymers, thickeners, film-forming agents, UV absorbers, metal ion sequestering agents, amino acids, organic amines, polymer emulsions, pH adjusters, skin nutrients, vitamins, antioxidants, antioxidant aids and perfumes, in amounts that do not inhibit the effects of the present disclosure.

The terms “powder” and “powdered component” as used herein are synonymous. The powder is not particularly limited so long as it is generally usable for cosmetic purposes, for example. Examples of the powder bodies may include inorganic powder (such as talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, tungstate, magnesium, silica, zeolite, glass, barium sulfate, calcined calcium sulfate (calcined gypsum), calcium phosphate, fluorine apatite, hydroxyapatite, ceramic powder, metallic soap (such as zinc myristate, calcium palimitate, and aluminum stearate), and boron nitride, etc); organic powder (such as polyamide resin powder (nylon powder), polyethylene powder, polymethylmethacrylate powder, polystyrene powder, styrene-acrylic acid copolymer powder, benzoguanamine resin powder, poly(tetrafluroethylene) powder, and cellulose powder, silicone resin powder, silk powder, wool powder, urethane powder, etc); inorganic white family pigment (such as titanium dioxide, zinc oxide, etc); inorganic red family pigment (such as iron oxide (colcothar), iron titanate, etc); inorganic brown family pigment (such as γ-iron oxide, etc); inorganic yellow family pigment (such as yellow iron oxide, loess, etc); inorganic black family pigment (such as black iron oxide, carbon black, lower titanium oxide, etc); inorganic purple family pigment (such as manganese violet, cobalt violet, etc); inorganic green family pigment (such as chrome oxide, chrome hydroxide, cobalt titanate, etc); inorganic blue family pigment (such as ultramarine, iron blue, etc); pearl pigment (such as titanium oxide coated mica, titanium oxide coated bismuth oxychloride, titanium oxide coated talc, colored titanium oxide coated mica, bismuth oxychloride, argentine, etc); metal powder pigment (such as aluminum powder, copper powder, etc); organic pigment such as zirconium, barium, or aluminum lake (such as organic pigment such as Red No. 201, Red No. 202, Red No. 204, Red No. 205, Red No. 220, Red No. 226, Red No. 228, Red No. 405, Red No. 201, Orange No. 203, Orange No. 204, Yellow No. 205, Yellow No. 401, Blue No. 401, Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, and Blue No. 1, etc); natural pigment (such as chlorophyll, (3-carotene, etc) and the like.

Examples of the moisturizers may include polyethylene glycol, propylene glycol, glycerin, 1,3-butylene glycol, xylitol, sorbitol, maltitol, chondroitin sulfate, hyaluronic acid, mucoitin sulfate, charonic acid, atelocollagen, cholesteryl 12-hydroxystearate, sodium lactate, bile salt, dl-pyrrolidone carboxylate, alkyleneoxide derivative, short-chain soluble collagen, diglycerin (EO)PO adduct, chestnut rose extract, yarrow extract, melilot extract, and the like.

Examples of the natural water-soluble polymer may include plant-based polymer (such as gum Arabic, gum tragacanth, galactan, guar gum, locust bean gum, gum karaya, carrageenan, pectine, agar, quince seed (Cydonia oblonga), algae colloid (brown algae extract), starch (rice, corn, potato, wheat), glicyrrhizic acid); microorganism based polymer (such as xanthan gum, dextran, succinoglycan, pullulan, etc), animal-based polymer (such as collagen, casein, albumin, gelatine, etc) and the like.

Examples of the semisynthetic water-soluble polymer may include starch-based polymer (such as carboxymethyl starch, methylhydroxypropyl starch, etc); cellulose-based polymer (such as methylcellulose, ethylcellulose, methylhydroxypropylcellulose, hydroxyethylcellulose, cellulose sodium sulfate, hydroxypropylcellulose, carboxymethylcellulose, sodium calboxymethyl cellulose, crystalline cellulose, cellulose powder, etc); algin acid-based polymer (such as sodium alginate, propylene glycol alginate ester, etc), and the like.

Examples of the synthetic water-soluble polymer may include vinyl based polymer (such as polyvinyl alcohol, polyvinyl methyl ether, polyvinylpyrrolidone, carboxyvinylpolymer, etc); polyoxyethylene based polymer (such as polyoxyethylenepolyoxypropylene copolymer such as polyethylene glycol 20,000, 40,000 and 60,000, etc); acrylic polymer (such as sodium polyacrylate, polyethylacrylate, polyacrylamide, etc); polyethyleneimine; cationic polymer; and the like.

Examples of other thickeners may include gum arabic, carrageenan, karaya gum, tragacanth gum, carob gum, quince seed (marmelo), casein, dextrin, gelatin, sodium pectate, sodium alginate, methyl cellulose, ethyl cellulose, carboxymethyl cellulose (CMC), hydroxyethyl cellulose, hydroxypropyl cellulose, polyvinyl alcohol (PVA), polyvinylmethyl ether (PVM), PVP (polyvinyl pyrrolidone), polysodium acrylate, carboxyvinyl polymer, locust bean gum, guar gum, tamarind gum, dialkyldimethylammonium sulfate cellulose, xanthan gum, aluminum magnesium silicate, bentonite, hectorite, aluminum magnesium silicate (Veegum), sodium magnesium silicate (Laponite), silicic acid anhydride, taurate-based synthetic polymers, and acrylate-based synthetic polymers.

Examples of the film-forming agent may include an anionic film-forming agent (such as (meta)acrylic acid/(meta)acrylic acid ester copolymer, methyl vinyl ether/maleic anhydride coplymer, etc), a cationic film-forming agent (such as cationic cellulose, diallyldimethylammonium chloride polymer, diallyldimethylammonium chloride/acrylic amide copolymer, etc), a nonionc film-forming agent (such as polyvinyl alcohol, polyvinylpyrrolidone, polyvinyl acetate, polyacrylic ester copolymer, (meta)acrylamide, polymeric silicone, silicone resin, trimethylsiloxysilicate, etc), and the like.

Examples of the ultraviolet light absorbers may include benzoic acid family ultraviolet light absorber (such as p-aminobenzoic acid (hereinafter abbreviated as PABA), PABA monoglycerine ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc); anthranilic acid family ultraviolet light absorber (such as homomenthyl N-acetylanthranilate etc); salicylic acid family ultraviolet light absorber (such as amyl salicylate, menthyl salicylate, homomenthyl salicylate, octyl salicylate, phenyl salicylate, benzyl salicylate, p-isopropanolphenyl salicylate, etc); cinnamic acid family ultraviolet light absorber (such as octyl methoxycinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxy cinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate, glyceryl mono-2-ethylhexanoyl-diparamethoxy cinnamate, etc); benzophenone family ultraviolet light absorber (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc); 3-(4′-methylbenzylidene)-d,l-camphor and 3-benzylidene-d,l-camphor; 2-phenyl-5-methylbenzoxazol; 2,2′-hydroxy-5-methylphenylbenzotriazol, 2-(2′-hydroxy-5′-t-octylphenyl) benzotriazol, 2-(2′-hydroxy-5′-methylphenylbenzotriazol; dibenzalazine; dianisoylmethane; 4-methoxy-4′-t-butyldibenzoylmethane; 5-(3,3-dimethyl-2-norbornylidene)-3-pentane-2-one; dimorpholinopyridazinone; 2-ethylhexyl 2-cyano-3,3-diphenylacrylate; 2,4-bis-{[4-(2-ethylhexyloxy)-2-hydroxy]-phenyl}-6-(4-methoxyphenyl)-(1,3,5)-triazine, and the like.

Examples of the metal ion sequestrant may include 1-hydroxyethane-1,1-diphosphonic acid, 1-hydroxyethane, 1-diphosphonic acid 4Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, trisodium hydroxyethyl ethylenediamine triacetate, and the like.

Examples of the amino acid may include neutral amino acid (such as threonine, cysteine, etc); basic amino acid (such as hydroxylysine, etc) and the like. Examples of the amino acid derivative may include sodium acyl sarcosinate (sodium lauroyl sarcosinate), acyl glutamate, sodium acyl β-alanine, glutathione, pyrrolidone carboxylate, and the like.

Examples of the organic amine may include monoethanolamine, diethanolamine, triethanolamine, morpholine, triisopropanolamine, 2-amino-2-methyl-1,3-propanediol, 2-amino-2-methyl-1-propanol, and the like.

Examples of the polymer emulsion may include acrylic resin emulsion, ethyl polyacrylate emulsion, solution of acrylic resin, polyacrylalkylester emulsion, polyvinyl acetate resin emulsion, natural rubber latex, and the like.

Examples of the pH modifier may include buffer such as lactic acid-sodium lactate, citric acid-sodium citrate, succinic acid-sodium succinate, and the like.

Examples of the vitamins may include vitamine A, B1, B2, B6, C, E and derivatives thereof, pantothenic acid and derivatives thereof, biotin, and the like.

Examples of the anti-oxidant may include tocopherols, dibutyl hydroxy toluene, butyl hydroxy anisole, and gallic acid esters, and the like.

Examples of the anti-oxidant aid may include phosphoric acid, citric acid, ascorbic acid, maleic acid, malonic acid, succinic acid, fumaric acid, cephalin, hexamethaphosphate, phytic acid, ethylenediaminetetraacetic acid, and the like.

Examples of other containable compositions may include an antiseptic agent (such as ethylparaben, butylparaben, chlorphenesin, 2-phenoxyethanol, etc); antiphlogistic (such as glycyrrhizinic acid derivatives, glycyrrhetic acid derivatives, salicylic acid derivatives, hinokitiol, zinc oxide, allantoin, etc); a skin-whitening agent (such as placental extract, saxifrage extract, arbutin, etc); various extracts (such as phellodendron bark (cork tree bark), coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, red pepper, citrus unshiu, Japanese angelica, seaweed, etc); an activator (such as royal jelly, photosenstizer, cholesterol derivatives, etc); a blood circulation promotion agent (such as nonylic acid vanillylamide, nicotine acid benzyl ester, nicotine acid β-butoxyethyl ester, capsaicin, zingerone, cantharides tincture, ichthammol, tannic acid, α-borneol, tocopheryl nicotinate, meso-inositol hexanicotinate, cyclandelate, cinnarizine, tolazoline, acetylcholine, verapamil, cepharanthine, γ-oryzanol, etc); an antiseborrheric agent, (such as sulfur, thianthl, etc); an anti-inflammatory agent (such as tranexamic acid, thiotaurine, hypotaurine, etc), and the like.

The composition of the present disclosure further may inculde, as necessary, caffeine, tannin, verapamil, tranexamic acid and derivatives thereof; various crude drug extracts such as licorice, Chinese quince, Pyrola japonica and the like; drugs such as tocopherol acetate, glycyrrhetinic acid, glycyrrhizic acid and derivatives thereof, or salts thereof; skin-whitening agents such as vitamin C, magnesium ascorbyl phosphate, ascorbic acid glucoside, arbutin, kojic acid and the like; amino acids such as arginine and lysine and the like and derivatives thereof.

The pH of the aqueous phase is preferably 5 or higher, more preferably 5.5 or higher. If the pH is below 5, emulsification after shaking may become insufficient. The pH of the aqueous phase is preferably 8 or lower, more preferably 7.5 or lower. If the pH exceeds 8, irritation to the skin may become too strong.

Appearance:

It is preferred that the interface between the aqueous phase and the oil phase is clear. Also, it is preferred that both the aqueous phase and oil phase are non-turbid, and are more preferably transparent.

Usability:

Methods for using the oil-water separating composition of the present disclosure will be described below. The oil-water separating composition can be suitably used, for example, for cosmetics, cleansing agents, and the like.

As cleansing agents, the composition can be suitably used, for example, for cosmetic removal cleansing agents, hand soaps, body soaps, hair cleansing agents, kitchen cleansing agents, and the like. Cosmetic cleansing agents may include, for example, cleansing agents for removing waterproof-type cosmetics (makeup). The cleansing composition can be used as a cleansing agent that is rinsed off with water, or can be used as a cleansing agent that is not rinsed off with water (i.e., a leave-on-type composition). In cases where the composition is a leave-on type composition, the cleansing composition can be, for example, applied or dropped onto an object being cleansed (e.g., the skin), and can then be wiped off with a fiber product such as a pad. Alternatively, a fiber product may be impregnated with the cleansing composition, and an object being cleansed (e.g., the skin) may be wiped therewith.

It is preferred that a container holding the oil-water separating composition of the present disclosure is shaken before extracting the composition to bring the composition into a temporarily emulsified state (including a quasi-emulsified state), and then the composition may be taken out from the container. The number of times to shake the container to bring the composition into an emulsified state may be, for example, preferably 20 times or fewer, more preferably 15 times or fewer, further preferably 10 times or fewer. It is preferred that the temporarily emulsified state created by the shaking operation lasts for a predetermined time. For example, it is preferred that the temporarily emulsified state lasts for preferably 10 seconds or longer, more preferably 15 seconds or longer, further preferably 20 seconds or longer. It is also preferred that the emulsified state returns to the original oil-water separated state by being left to stand. It is preferred that, when the oil-water separating composition returns from the temporarily emulsified state to the oil-water two-layer separated state by being left standing, the composition is not turbid but is transparent, and it is also preferred that the interface between the oil phase and the aqueous phase is clear.

The oil-water separating composition of the present disclosure can inhibit liquid droplets from continuing to adhere to the inner wall surface of a container above the liquid surface for a long period of time. Stated differently, with the oil-water separating composition of the present disclosure, even if liquid droplets are formed on the inner wall surface of a container, the liquid droplets can be made to disappear spontaneously in a short time. Thus, it is possible to maintain excellent aesthetic appearance of a container containing the oil-water separating composition.

Manufacturing Method:

A method for manufacturing the oil-water separating composition of the present disclosure will be described below. The method for manufacturing the oil-water separating composition of the present disclosure is not particularly limited, and the composition can be prepared by generally known methods. For example, the oil-water separating composition can be prepared by mixing the aforementioned components. The oil-water separating composition can be manufactured by preparing the aqueous phase and the oil phase separately, and then mixing the aqueous phase and the oil phase. In this case, the polyol derivative may be added to the oil phase. Alternatively, all of the components may be mixed at once, without separately preparing the oil phase and the aqueous phase.

There may be cases where it is difficult, or utterly impractical, to directly define the phase constitution etc. of the oil-water separating composition of the present disclosure based on the compositional makeup thereof. In such circumstances, it should be permissible to define the oil-water separating composition of the present disclosure according to methods for producing the same.

An article according to a second embodiment will be described.

An article according to the present disclosure includes: the oil-water separating composition according to the first embodiment; and a container that houses the oil-water separating composition. At least a portion of the container has transparency enabling an interior of the container to be visible. Particularly, it is preferred that the container has transparency enabling the separated state and emulsified state of the oil-water separating composition to be visually observable from the outside. The container may be colorless or colored.

Examples of materials for the container may include resins, glass, and the like. The material for the container is preferably a resin which is lightweight and enables the container to be easily shaken with the hand. It is preferred that the container contains a resin having a polyester as a basic skeleton, such as polyethylene terephthalate (PET).

It is preferred that the container has a structure that does not cause liquid leakage even when shaken with the oil-water separating composition housed therein. It is preferred that the container includes: a body for housing the oil-water separating composition, and having an opening from which the oil-water separating composition can be taken out; and a lid or a cap capable of opening/closing the opening.

The article of the present disclosure enables the oil-water separating composition according to the first embodiment to be taken out from the container after confirming the emulsified state of the oil-water separating composition. Thus, the oil-water separating composition can be used in a more suitable state. Further, by using a resin-made container, the oil-water separating composition can be emulsified easily by a manual shaking operation.

In the article of the present disclosure, adhesion of liquid droplets on the container's inner surface above the oil-water separating composition is inhibited. Also, when the oil-water separating composition is in a separated state, the interface between the oil phase and the aqueous phase can be made clear.

### EXAMPLES

The oil-water separating composition of the present disclosure will be described below by way of examples. The oil-water separating composition of the present disclosure is, however, not limited to the following examples. The following describes examples wherein the oil-water separating compositions according to the respective Test Examples are used for cosmetic cleansing agents, but the composition of the present disclosure is not limited to cosmetic cleansing agents. The unit employed for indicating the content by percentage of each component shown in the Tables is percent by mass (mass %).

### Test Examples 1 to 3

Oil-water two-layer separation-type cleansing compositions were prepared. Table 1 shows the compositional makeup and evaluation results for each cleansing composition prepared in the respective Test Examples. The oil-water two-layer-type cleansing composition was prepared by first preparing the oil phase and the aqueous phase separately, and then mixing the two phases. In the tables below, the “oil phase” and the “aqueous phase” indicate the respective phases to which the various components were blended at the time of preparation of the cleansing composition. It should thus be added that, by shaking and/or during standing (in a stationary state), components blended to the oil phase may migrate to the aqueous phase, and/or components blended to the aqueous phase may migrate to the oil phase.

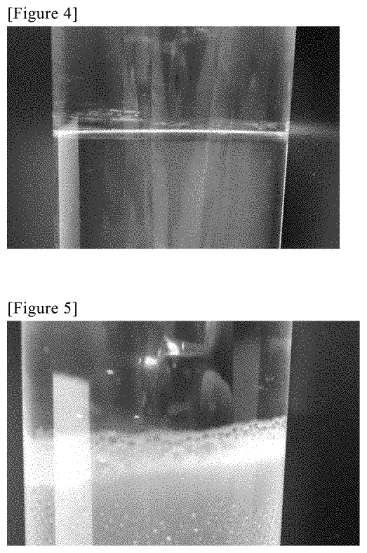

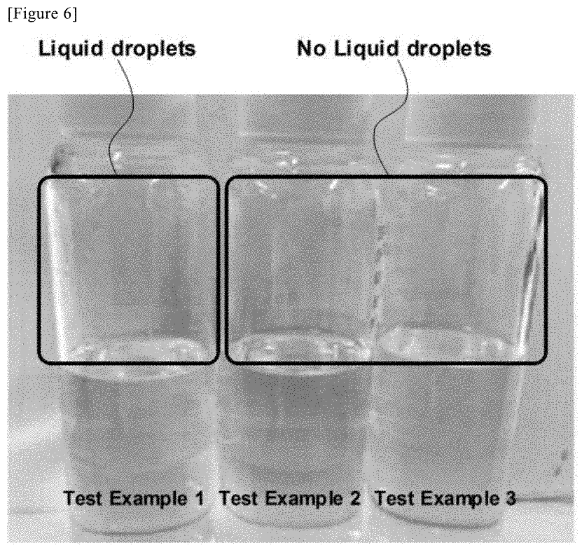

Each prepared oil-water separation-type cleansing composition was placed in a transparent sample tube made of polyethylene terephthalate (PET). In a state left to stand for 3 hours, the cleansing composition in the sample tube was separated into two layers, with the oil phase constituting the upper layer and the aqueous phase constituting the lower layer. In a state where the oil phase and the aqueous phase were separated into two layers, the container was shaken manually 8 times in the vertical direction, to emulsify the oil phase and the aqueous phase. After emulsification, the container was left to stand for 3 hours, to make the oil phase and the aqueous phase re-separate. The state of liquid droplet adhesion on the container's inner wall surface in a space above the liquid surface was visually observed before and after emulsification, and was evaluated according to the following criteria. The state of the interface and the state of each phase were visually observed before and after emulsification, and were evaluated according to the following criteria. FIGS. 1 to 3 show photographs of a sample tube, respectively showing examples of evaluation states for the residual liquid droplet evaluation. FIGS. 4 and 5 show photographs of a sample tube, respectively showing examples of evaluation states for the oil phase/liquid phase interface evaluation. The duration of the emulsified state was evaluated according to the following criteria.

Liquid Droplet Adhesion:

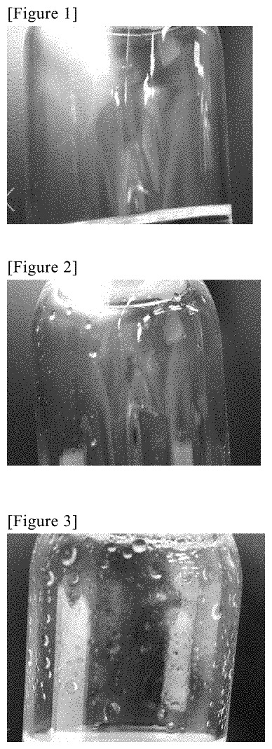

A: No liquid droplet remained on the container's inner wall surface (see FIG. 1).
B: Liquid droplets slightly remained on the container's inner wall surface (see FIG. 2).
C: Large liquid droplets remained on the container's inner wall surface (see FIG. 3).

State of Interface and Presence/Absence of Turbidity in Each Layer:

A: The interface between the oil phase and the liquid phase was clear (see FIG. 4), and neither the oil phase nor the aqueous phase was turbid.
B: The interface between the oil phase and the liquid phase was somewhat unclear, or either the oil phase or the aqueous phase was somewhat turbid.
C: The interface between the oil phase and the liquid phase was apparently unclear (see FIG. 5), or either the oil phase or the aqueous phase was apparently turbid.

Emulsification Time:

A: The duration of the emulsified state was 10 seconds or longer.
B: The duration of the emulsified state was less than 10 seconds.

FIG. 6 shows a photograph of oil-water two-layer-type cleansing compositions according to Test Examples 1 to 3. The samples shown in FIG. 6 are cleansing compositions in a state where the two layers have separated by being left standing for 3 hours after the shaking process.

In Test Example 1 which contained no polyol derivative, it was observed that liquid droplets continued to adhere in a dispersed state to the entire inner wall surface of the container. In contrast, in Test Examples 2 and 3 which contained a polyol derivative, no liquid droplets adhering to the inner wall surface were observed. Formation of liquid droplets on the container's inner wall surface was observed immediately after the shaking process, but the liquid droplets disappeared spontaneously immediately after being left standing for several seconds. This suggests that the polyol derivative has, in the oil-water separating composition, an action of preventing adhesion of liquid droplets.

Further, in Test Example 1, the interface between the oil phase and the aqueous phase became unclear after emulsification. In contrast, in Test Examples 2 and 3, before emulsification, the interface between the oil phase and the aqueous phase was clear, and no turbidity, such as white turbidity, was observed in either phase. In an emulsified state created by shaking, it was possible to keep the emulsified state lasting for a time sufficient for use. Also, in a re-separated state after the emulsified state, the interface was clear and no turbidity was observed in either phase, and the composition was able to return to the same state as before being shaken. This suggests that the polyol derivative has an action of clarifying the interface after the temporarily emulsified state of the oil-water separating composition.

TABLE 1

| Test Example | | | 1 | 2 | 3 |
|---|---|---|---|---|---|
| Oil phase | Volatile dimethicone | | 24 | 23.8 | 23 |
| | Isododecane | | 8 | 8 | 8 |
| | Ethylhexylglycerin | | — | 0.2 | 1 |
| Aqueous | Ethanol | | 3 | 3 | 3 |
| phase | 1,3-Butylene glycol | | 8 | 8 | 8 |
| | Lauryldimethylaminoacetic acid betaine | | 0.05 | 0.05 | 0.05 |
| | Sodium chloride | | 0.5 | 0.5 | 0.5 |
| | Phenoxyethanol | | 0.2 | 0.2 | 0.2 |
| | Citric acid | | 0.01 | 0.01 | 0.01 |
| | Sodium citrate | | 0.09 | 0.09 | 0.09 |
| | EDTA-2Na•2H₂O | | 0.02 | 0.02 | 0.02 |
| | Ion-exchanged water | | Balance | Balance | Balance |
| Total | | | 100 | 100 | 100 |
| Evaluation | pH | | 5.91 | 5.94 | — |
| | After | Liquid droplet adhesion | C | A | A |
| | emulsification | Interface clarity and presence/ | B | A | A |
| | | absence of turbidity | | | |
| | Before | Emulsification time | A | A | A |
| | emulsification | Liquid droplet adhesion | C | A | A |
| | | Interface clarity and presence/ | A | A | A |
| | | absence of turbidity | | | |

### Test Examples 4 to 10

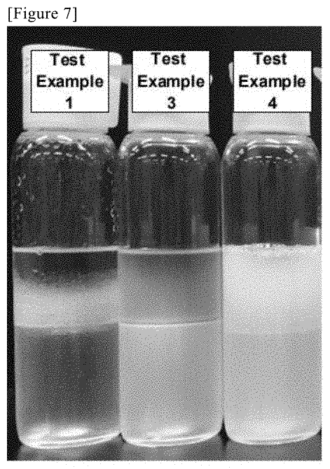

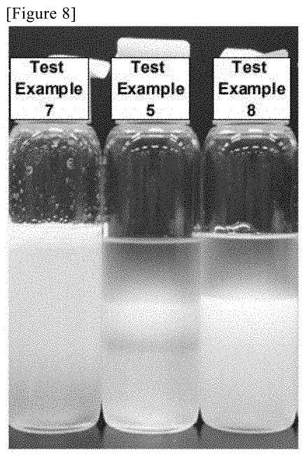

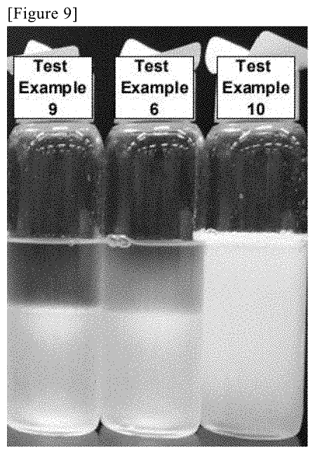

In Test Examples 4 to 10, compounds other than ethylhexylglycerin were evaluated to see whether they had the aforementioned liquid droplet adhesion inhibition effect and interface clarifying effect. The evaluation methods were the same as those for Test Examples 1 to 3. The evaluation criteria for liquid droplet adhesion were the same as the aforementioned evaluation criteria. The evaluation criteria for interface clarity were the same as the aforementioned evaluation criteria, except that the rating B was not used and only ratings A and C were used. Table 2 shows the compositional makeup and evaluation results. FIGS. 7 to 9 show photographs of oil-water two-layer-type cleansing compositions after shaking according to Test Examples 1 and 3 to 10.

Test Example 4, which used hexylglycerin, was capable of preventing adhesion of liquid droplets to the container's inner wall surface and was also capable of clarifying the interface between the oil phase and the aqueous phase, as in Test Examples 2 and 3. This suggests that glycerin derivatives represented by the aforementioned Chem. 3 have the aforementioned liquid droplet adhesion inhibition effect and interface clarifying effect.

Further, Test Examples 5 and 6, which respectively used propylene glycol laurate and propylene glycol isostearate, also exhibited the liquid droplet adhesion inhibition effect and interface clarifying effect. This suggests that propylene glycol mono-fatty acid esters as represented by the aforementioned Chem. 4 have the aforementioned liquid droplet adhesion inhibition effect and interface clarifying effect.

TABLE 2

| Test Example | | 4 | 5 | 6 | 7 | 8 | 9 | 10 |
|---|---|---|---|---|---|---|---|---|
| Oil phase | Volatile dimethicone | 23 | 23 | 23 | 23 | 23 | 23 | 23 |
| | Isododecane | 8 | 8 | 8 | 8 | 8 | 8 | 8 |
| | Hexylglycerin | 1 | — | — | — | — | — | — |
| | Propylene glycol | — | 1 | — | — | — | — | — |
| | laurate | | | | | | | |
| | Propylene glycol | — | — | 1 | — | — | — | — |
| | isostearate | | | | | | | |
| | PPG/PPG-14/7 | — | — | — | 1 | — | — | — |
| | dimethyl ether | | | | | | | |
| | Isostearic acid | — | — | — | — | 1 | — | — |
| | Isostearyl alcohol | — | — | — | — | — | 1 | — |
| | Propylene glycol | — | — | — | — | — | — | 1 |
| | dicaprate | | | | | | | |
| Aqueous | Ethanol | 3 | 3 | 3 | 3 | 3 | 3 | 3 |
| phase | 1,3-Butylene glycol | 8 | 8 | 8 | 8 | 8 | 8 | 8 |
| | Lauryldimethylaminoacetic | 0.05 | 0.05 | 0.05 | 0.05 | 0.05 | 0.05 | 0.05 |
| | acid betaine | | | | | | | |
| | Sodium chloride | 0.5 | 0.5 | 0.5 | 0.5 | 0.5 | 0.5 | 0.5 |
| | Phenoxyethanol | 0.2 | 0.2 | 0.2 | 0.2 | 0.2 | 0.2 | 0.2 |
| | Citric acid | 0.01 | 0.01 | 0.01 | 0.01 | 0.01 | 0.01 | 0.01 |
| | Sodium citrate | 0.09 | 0.09 | 0.09 | 0.09 | 0.09 | 0.09 | 0.09 |
| | EDTA-2Na•2H₂O | 0.02 | 0.02 | 0.02 | 0.02 | 0.02 | 0.02 | 0.02 |
| | Ion-exchanged water | Balance | Balance | Balance | Balance | Balance | Balance | Balance |
| Total | | 100 | 100 | 100 | 100 | 100 | 100 | 100 |
| Evaluation | Liquid droplet | A | B | B | C | A | C | C |
| | adhesion | | | | | | | |
| | Interface clarity | A | A | A | C | C | C | C |

### Test Examples 11 to 16

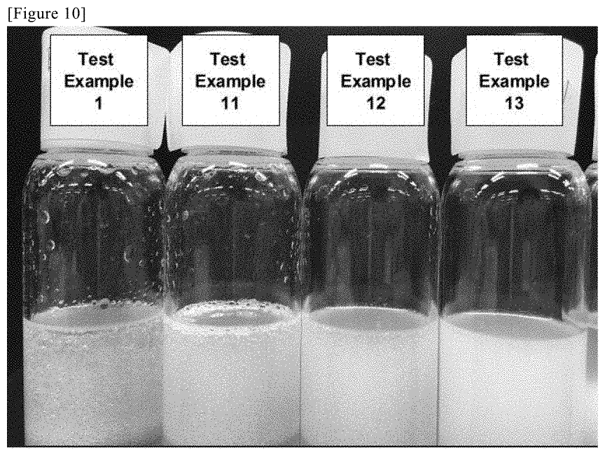

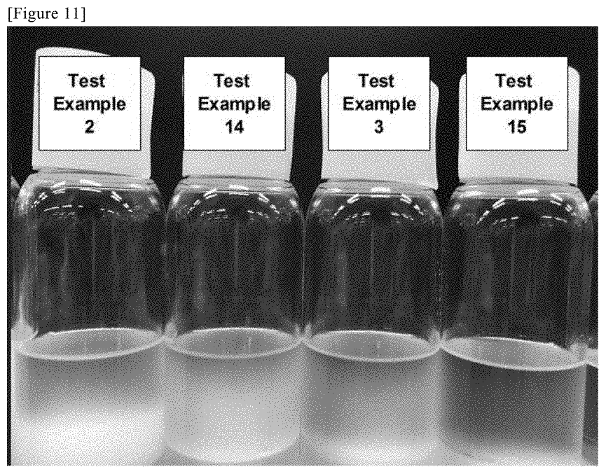

In Test Examples 11 to 16, the content by percentage of the polyol derivative was varied. The evaluation methods were the same as those for Test Examples 1 to 3. The evaluation criteria for liquid droplet adhesion were the same as the aforementioned evaluation criteria. The evaluation criteria for interface clarity were the same as the aforementioned evaluation criteria, except that the rating B was not used and only ratings A and C were used. Table 3 shows the compositional makeup and evaluation results. FIGS. 10 and 11 show photographs of oil-water two-layer-type cleansing compositions after shaking according to Test Examples 1 to 3 and 11 to 15.

In Test Example 11 and 12 wherein the content by percentage of the polyol derivative was low, it was observed that liquid droplets remained on the container's inner wall and the interface between the oil phase and the aqueous phase was unclear. In contrast, in Test Examples 2, 3, and 13 to 16 which contained at least 0.1% by mass of the polyol derivative, no adhesion of liquid droplets on the container's inner wall was observed. On the other hand, in Test Examples 15 and 16 which contained 2% by mass or greater of the polyol derivative, it was observed that the composition separated into three layers at the time of re-separation.

This suggests that the content by percentage of the polyol derivative with respect to the mass of the composition is preferably 0.06% by mass or greater, more preferably 0.07% by mass or greater, further preferably 0.08% by mass or greater. This also suggests that the content by percentage of the polyol derivative with respect to the mass of the composition is preferably 1.8% by mass or less, more preferably 1.5% by mass or less, further preferably 1.2% by mass or less.

TABLE 3

| Test Example | | 11 | 12 | 13 | 14 | 15 | 16 |
|---|---|---|---|---|---|---|---|
| Oil phase | Volatile dimethicone | 24 | 24 | 24 | 24 | 22.5 | 20.2 |
| | Isododecane | 8 | 8 | 8 | 8 | 7.5 | 6.8 |
| | Ethylhexylglycerin | 0.05 | 0.075 | 0.1 | 0.5 | 2 | 5 |
| Aqueous | Ethanol | 3 | 3 | 3 | 3 | 3 | 3 |
| phase | 1,3-Butylene glycol | 8 | 8 | 8 | 8 | 8 | 8 |
| | Lauryldimethylaminoacetic | 0.05 | 0.05 | 0.05 | 0.05 | 0.05 | 0.05 |
| | acid betaine | | | | | | |
| | Sodium chloride | 0.5 | 0.5 | 0.5 | 0.5 | 0.5 | 0.5 |
| | Phenoxyethanol | 0.2 | 0.2 | 0.2 | 0.2 | 0.2 | 0.2 |
| | Citric acid | 0.01 | 0.01 | 0.01 | 0.01 | 0.01 | 0.01 |
| | Sodium citrate | 0.09 | 0.09 | 0.09 | 0.09 | 0.09 | 0.09 |
| | EDTA-2Na•2H₂O | 0.02 | 0.02 | 0.02 | 0.02 | 0.02 | 0.02 |
| | Ion-exchanged water | Balance | Balance | Balance | Balance | Balance | Balance |
| Total | | 100 | 100 | 100 | 100 | 100 | 100 |
| Evaluation | Liquid droplet adhesion | C | B | A | A | A | A |
| | Interface clarity | C | C | A | A | C | C |

### Test Examples 17 to 20

In Test Examples 17 and 18, the same tests as in the aforementioned Test Examples were conducted for compositions not containing an amphoteric surfactant. In Test Examples 19 and 20, the same tests as in the aforementioned Test Examples were conducted for compositions not containing an inorganic salt. The evaluation methods and evaluation criteria were the same as those for Test Examples 4 to 10. Table 4 shows the compositional makeup and evaluation results.

A comparison between Test Example 1 and Test Example 17 shows that adding an amphoteric surfactant makes liquid droplets more prone to adhere to the container's inner wall surface, and also makes the interface between the oil phase and the aqueous phase prone to become turbid. In contrast, by adding a glycerin derivative as in Test Example 18, it was possible to ameliorate liquid droplet adhesion compared to the composition of Test Example 17 which did not contain an amphoteric surfactant. This shows that the liquid droplet adhesion inhibition effect is attributable to the polyol derivative.

A comparison between Test Example 1 and Test Example 19 shows that adding an inorganic salt makes liquid droplets more prone to adhere to the container's inner wall surface. In contrast, by adding a glycerin derivative as in Test Example 20, it was possible to ameliorate liquid droplet adhesion and improve interface clarity compared to the composition of Test Example 19 which did not contain an inorganic salt. This shows that the liquid droplet adhesion inhibition effect is attributable to the polyol derivative.

TABLE 4

| Test Example | | 17 | 18 | 19 | 20 |
|---|---|---|---|---|---|
| Oil phase | Volatile dimethicone | 24 | 24 | 24 | 24 |
| | Isododecane | 8 | 8 | 8 | 8 |
| | Ethylhexylglycerin | — | 0.2 | — | 0.2 |
| Aqueous | Ethanol | 3 | 3 | 3 | 3 |
| phase | 1,3-Butylene glycol | 8 | 8 | 8 | 8 |
| | Lauryldimethylaminoacetic acid betaine | — | — | 0.05 | 0.05 |
| | Sodium chloride | 0.5 | 0.5 | — | — |
| | Phenoxyethanol | 0.2 | 0.2 | 0.2 | 0.2 |
| | Citric acid | 0.01 | 0.01 | 0.01 | 0.01 |
| | Sodium citrate | 0.09 | 0.09 | 0.09 | 0.09 |
| | EDTA-2Na•2H₂O | 0.02 | 0.02 | 0.02 | 0.02 |
| | Ion-exchanged water | Balance | Balance | Balance | Balance |
| Total | | 100 | 100 | 100 | 100 |
| Evaluation | Liquid droplet adhesion | B | A | B | A |
| | Interface clarity | A | A | A | A |

### Test Examples 21 to 24

In Test Examples 21 to 24, decyltetradecyl dimethylamine oxide, i.e. a trialkylamine oxide, was added to the composition according to Test Example 2 to improve cleansability against dyes. In Test Examples 21 and 22, decyltetradecyl dimethylamine oxide was added to the oil phase, and in Test Examples 23 and 24, decyltetradecyl dimethylamine oxide was added to the aqueous phase. Also in Test Example 24, lauryldimethylaminoacetic acid betaine, i.e. an amphoteric surfactant, was added to the oil phase. Table 4 shows the compositional makeup and evaluation results for each cleansing composition. The evaluation criteria were the same as those for Test Examples 1 to 3.

In Test Example 21 which contained no polyol derivative, adhesion of liquid droplets to the container's inner wall was observed, as in Test Example 1. In contrast, in Test Examples 22 to 24 which contained a polyol derivative, no liquid droplet adhesion was observed. This shows that, even when a trialkylamine oxide was added, there was no change in the polyol derivative's liquid droplet inhibition effect.

In Test Example 21, white turbidity in the aqueous phase was observed after the emulsification process. In contrast, no white turbidity was observed in Test Examples 22 and 23. White turbidity in Test Example 21 is thought to be caused by the absence of the polyol derivative or insufficiency in the amount of ethanol.

TABLE 5

| Test Example | | | 21 | 22 | 23 | 24 |
|---|---|---|---|---|---|---|
| Oil phase | Volatile dimethicone | | 24 | 23.8 | 23.8 | 23.8 |
| | Isododecane | | 8 | 8 | 8 | 8 |
| | Ethylhexylglycerin | | — | 0.2 | 0.2 | 0.2 |
| | Decyltetradecyl dimethylamine oxide | | — | — | 1 | 1 |
| | Lauryldimethylaminoacetic acid betaine | | — | — | — | 0.05 |
| Aqueous | Ethanol | | 3 | 8 | 8 | 8 |
| phase | 1,3-Butylene glycol | | 8 | 8 | 8 | 8 |
| | Decyltetradecyl dimethylamine oxide | | 1 | 1 | — | — |
| | Lauryldimethylaminoacetic acid betaine | | 0.05 | 0.05 | 0.05 | — |
| | Sodium chloride | | 0.5 | 0.5 | 0.5 | 0.5 |
| | Phenoxyethanol | | 0.2 | 0.2 | 0.2 | 0.2 |
| | Citric acid | | 0.01 | 0.01 | 0.01 | 0.01 |
| | Sodium citrate | | 0.09 | 0.09 | 0.09 | 0.09 |
| | EDTA-2Na•2H₂O | | 0.02 | 0.02 | 0.02 | 0.02 |
| | Ion-exchanged water | | Balance | Balance | Balance | Balance |
| Total | | | 100 | 100 | 100 | 100 |
| Evaluation | pH | | 6.43 | 6.43 | 6.39 | 6.41 |
| | After | Liquid droplet adhesion | C | A | A | A |
| | emulsification | Interface clarity and presence/ | A | A | A | A |
| | | absence of turbidity | | | | |
| | Before | Emulsification time | A | A | A | A |
| | emulsification | Liquid droplet adhesion | C | A | A | A |
| | | Interface clarity and presence/ | C | A | A | A |
| | | absence of turbidity | | | | |

The oil-water separating composition and article of the present invention have been described according to the foregoing embodiments and examples, but the invention is not limited to the foregoing embodiments and examples and may encompass various transformations, modifications, and improvements made to the various disclosed elements (including elements disclosed in the Claims, Description, and Drawings) within the scope of the invention and according to the fundamental technical idea of the present invention. Further, various combinations, substitutions, and selections of the various disclosed elements are possible within the scope of the claims of the invention.

Further issues, objectives, and embodiments (including modifications) of the present invention are revealed also from the entire disclosure of the invention including the Claims.

The numerical ranges disclosed herein are to be construed in such a manner that arbitrary numerical values and ranges falling within the disclosed ranges are treated as being concretely described herein, even where not specifically stated.

### INDUSTRIAL APPLICABILITY

Some or all of the foregoing embodiments may be described as in the following additional items, although not limited thereto. The various additional items may be employed in combination with the claim(s) in the Scope of Claims.

{Additional Item 1}

A method of use of the cleansing composition of the present disclosure, comprising applying the composition to a cosmetic.

{Additional Item 2}

A method of use of the cleansing composition of the present disclosure, comprising applying the composition for cleansing the skin.

{Additional Item 3}

A method of use of the cleansing composition of the present disclosure, comprising using the composition for removing a cosmetic.

{Additional Item 4}

A method of use of the cleansing composition of the present disclosure, comprising using the composition as a leave-on-type composition.

{Additional Item 5}

A method of use of the cleansing composition of the present disclosure, comprising shaking the cleansing composition into an emulsified state, and then using the composition.

{Additional Item 6}

A method of use of the cleansing composition of the present disclosure, comprising the cleansing composition containing a trialkylamine oxide to remove a dye from the skin.

### INDUSTRIAL APPLICABILITY

The oil-water separating composition of the present disclosure can be applied for cosmetics, cleanser, and so on to be applied to the skin. Particularly, the oil-water separating composition of the present disclosure can be suitably used for cleansing to remove cosmetics on the skin.

1. An oil-water separating composition comprising:
an aqueous phase; and
an oil phase separated from the aqueous phase in a stationary state, wherein:
the composition contains from 0.06 to 1.8% by mass of a polyol derivative with respect to the mass of the composition; and
the polyol derivative is a glycerin derivative represented by Chem. 1 and/or a glycol derivative represented by Chem. 2: 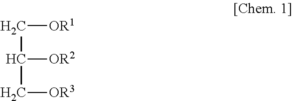
(in the chemical formula represented by Chem. 1, one of R¹, R², and R³ is a C₄₋₁₅ alkyl group, alkenyl group or acyl group, and two are each a hydrogen atom); 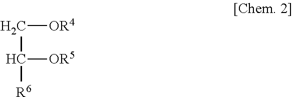
(in the chemical formula represented by Chem. 2, one of R⁴ and R⁵ is a C₁₀₋₂₀ alkyl group, alkenyl group or acyl group, and the other is a hydrogen atom; and R⁶ is a C₁₋₄ alkyl group, alkenyl group or acyl group, or a hydrogen atom).

2. The composition according to claim 1, further comprising from 0.01 to 0.1% by mass of an alkylbetaine-type surfactant.

3. The cleansing composition according to claim 1, wherein a content by percentage of a surfactant is 0.2% by mass or less with respect to the mass of the composition.

4. The composition according to claim 1, further comprising from 0.1 to 5% by mass of a salt with respect to the mass of the composition.

5. The cleansing composition according to claim 1, wherein the glycerin derivative includes ethylhexylglycerin and/or hexylglycerin.

6. The cleansing composition according to claim 1, wherein the glycol derivative includes at least one selected from the group consisting of propylene glycol laurate, propylene glycol stearate, and propylene glycol isostearate.

7. The cleansing composition according to claim 1, wherein:
a content by percentage of the oil phase is from 20 to 80% by mass with respect to the mass of the composition; and
a content by percentage of the aqueous phase is from 20 to 80% by mass with respect to the mass of the composition.

8. The composition according to any one of claims 1 to 7 claim 1, wherein the composition is to be used by being formed into a temporarily emulsified state by a user.

9. The composition according to claim 1, wherein the composition is a cosmetic.

10. The composition according to claim 1, wherein the composition is a cosmetic cleansing agent.

11. The composition according to claim 1, wherein the composition is a leave-on-type composition.

12. An article comprising:
the composition according to claim 1; and
a container that houses the composition, wherein
at least a portion of the container has transparency enabling an interior of the container to be visible.

13. The article according to claim 12, wherein the container comprises a polyester.

14. The cleansing composition according to claim 2, wherein a content by percentage of a surfactant is 0.2% by mass or less with respect to the mass of the composite

15. The cleansing composition according to claim 14, wherein:
a content by percentage of the oil phase is from 20 to 80% by mass with respect to the mass of the composition; and
a content by percentage of the aqueous phase is from 20 to 80% by mass with respect to the mass of the composition.

16. The composition according to claim 15, wherein the composition is to be used by being formed into a temporarily emulsified state by a user.

17. The composition according to claim 16, further comprising from 0.1 to 5% by mass of a salt with respect to the mass of the composition.

18. The cleansing composition according to claim 17, wherein the glycerin derivative includes ethylhexylglycerin and/or hexylglycerin.

19. The cleansing composition according to claim 17, wherein the glycol derivative includes at least one selected from the group consisting of propylene glycol laurate, propylene glycol stearate, and propylene glycol isostearate.

20. The composition according to claim 17, wherein the composition is a leave-on-type composition.
